(19) United States
(12) Patent Application Publication
(10) Pub. No.: US 2013/0092196 A1
(43) Pub. Date: Apr. 18, 2013

(54) METHOD FOR STERILIZING AND WASHING A CONTAINER

(71) Applicants: Nobuaki Nagatani, Tokyo (JP); Takeshi Iwashita, Yokohama-shi (JP); Youichi Kawasaki, Tokyo (JP)

(72) Inventors: Nobuaki Nagatani, Tokyo (JP); Takeshi Iwashita, Yokohama-shi (JP); Youichi Kawasaki, Tokyo (JP)

(73) Assignee: TOYO SEIKAN KAISHA, LTD., Tokyo (JP)

(21) Appl. No.: 13/704,527

(22) PCT Filed: Jun. 1, 2011
(86) PCT No.: PCT/JP2011/062573

§ 371 Date: Dec. 14, 2012

(30) Foreign Application Priority Data
Jun. 16, 2010 (JP) 2010-137455

(51) Int. Cl. A61L 2/18

(52) U.S. Cl. CPC A61L 2/18
USPC 134/26; 134/94.1

(57) ABSTRACT

The purpose of the present invention is to provide a container sterilization method, washing method, and system which retain food hygiene manufacturing standards and a required production line speed, as well as reducing the amount of treatment fluids used such as pharmaceutical fluid, hot water and aseptic water. The container sterilization method and washing method of the present invention is a method in which a container is sterilized/washed using a treatment fluid, and the sterilization step and/or washing step are divided into at least two parts. By storing and pumping upwards the treatment fluid supplied from one part of the divided sterilization or washing step after use, the treatment fluid can be supplied in series to the other part of the same divided sterilization or washing step.

### TECHNICAL FIELD

The present invention relates to a method for sterilizing and washing a container in what is called an aseptic filling technique for filling and sealing a beverage in a sterile environment.

### BACKGROUND

Because microorganisms grow more easily in a low-acid beverage such as green tea, oolong tea, red tea, coffee, barley tea, mixed tea, cocoa, milk tea, milk coffee, and milk shake than in a carbonated beverage and an acidic beverage, the low-acid beverage needs to be manufactured with the greatest care about contamination by and development of the microorganisms. Incidentally, according to manufacturing standards of Food Sanitation Act related to sterilization conditions for soft drinks, a sterilization condition for the low-acid beverage is at 120° C. for four minutes or stricter and is stricter than a sterilization condition for the acidic beverage with a pH lower than 4.0 which is at 65° C. for ten minutes or stricter and a sterilization condition for the acidic beverage with a pH not lower than 4.0 and lower than 4.6 which is at 85° C. for thirty minutes or stricter. Therefore, to fill the low-acid beverage or the like into a container which is not a can, i.e., into a not-heat-resistant container such as a plastic bottle made of polyester resin or the like, a bag-in-box, and a paper container, an aseptic filling method is employed. In the aseptic filling method, the container is sterilized in advance while the beverage is sterilized by heating under required conditions, cooled, and then filled and packed in the not-heat-resistant container in an aseptic environment, because the container is not resistant to hot packing or a retort treatment satisfying sterilization conditions required by the Food Sanitation Act.

As a method of sterilizing the plastic bottle, there are a method in which the bottle is sterilized with treatment solution including a medicine such as peracetic acid solution, hydrogen peroxide solution, sodium hypochlorite, and ozone water and then washed with aseptic water and a method in which the bottle is sterilized with hot water warmed to 65 to 85° C. without using a medicine and washed with aseptic water as necessary. Either one of the methods for sterilizing or washing the bottle is determined according to the kind of beverage. In each of these methods, amounts of medicinal solution, ozone water, hot water, aseptic water, and the like to be used are determined by required sterilization and washing times. As a speed of a line increases, an amount of the treatment solution to be supplied to the line increases and supply equipment such as a tank and a pump increases in size. Not only that, a length, i.e., an area of the line occupied by a sterilization washing step which requires a predetermined time increases and a system itself increases in size. As a result, an initial cost and a running cost increase.

As a solution for the problem of the increase in size of washing equipment due to the above-described increase in speed, separation of the washing step is proposed in Patent Literature 1. To achieve the object, in a container conveying method in a filling installation as shown in FIG. 3, when the container is washed with medicinal solution while retained and turned at least on a turntable A on an entrance side and is finish-washed while retained and turned on a turntable C on an exit side in a container washing portion of the filling installation for finish washing the container, filling liquid into the container, and capping the container, the container is inverted at an entrance of the container washing portion and fed to the turntable B on the entrance side, washed in an inverted attitude, moved in the inverted attitude between the turntables, discharged in the inverted attitude from the turntable on the exit side, and then inverted into an erected attitude. With the above-described structure, because the container is washed and moved between the turntables while retained in the inverted attitude from the entrance to the exit of the washing portion, entry of contaminants into the container can be prevented, a sufficient washing section can be obtained along a circling route of the turntables, and it is possible to adapt to high-speed washing.

However, this document discloses a structure in which the washing with the medicinal solution is carried out on the two turntables and the finish washing is carried out on the one turntable. Because the finish washing is carried out on the one turntable, washing water is naturally supplied from one tank while the medicinal agent to be supplied to the two turntables is supplied parallel from one tank by using a common pump. Because of this structure, in order to adapt to the increase in speed of the system, an amount of the washing water and an amount of the medicine become large and the tanks and the supply pumps increase in size. Therefore, it is impossible to solve the above-described large problem, i.e., increase of the initial cost and the running cost.

### CITATION LIST

Patent document 1: Japanese Patent Application Laid-Open No. 2001-240189, “CONTAINER CONVEYING METHOD IN FILLING INSTALLATION AND ROTARY CONTAINER WASHING INSTALLATION”, Date of Publication: Sep. 4, 2001

Patent document 2: Japanese Utility Model Application Publication No. 3-54689, “WASHING APPARATUS”, Date of Publication of Application: Dec. 3, 1991

### SUMMARY OF INVENTION

### Problem to be Solved by the Invention

It is an object of the present invention to provide a method for sterilizing and washing a container and a system for the method, in which an amount of treatment solution such as medicinal solution, hot water, and aseptic water to be used is suppressed while meeting manufacturing standards of Food Sanitation Act and securing a required speed of a production line.

### Means for Solving the Problem

A method for sterilizing and washing according to the present invention is a method of sterilizing and washing a container with treatment solution, wherein a sterilization step and/or a washing step determined by a sterilization or washing time are/is divided into two or more, the treatment solution supplied to a division of the sterilization or washing step is stored after use and pumped up to thereby be supplied in series to the other divisions of the same sterilization step and/or the washing step.

According to an aspect, the treatment solution supplied to a most downstream division of the sterilization step and/or the washing step is stored after use and pumped up, the treatment solution supplied to an adjacent upstream division of the same step is stored again and pumped up, and the treatment solution is successively supplied to the upstream divisions of the same step, so that the treatment solution is supplied in series to the all divisions of the step.

In the method for sterilizing and washing the container according to the invention, the kind of the treatment solution and the number of divisions of the sterilization step and the number of divisions of the washing step are selected according to the kind of beverage liquid.

In a system for sterilizing and washing a container according to the invention, a sterilizing zone and/or a washing zone determined by a sterilizing or washing time are/is divided into two or more, a tank and a pump for supplying treatment solution to each division of the sterilization zone and/or washing zone are disposed, and, to the tanks for supplying the treatment solution to the respective zones excluding the one tank for supplying the treatment solution first, the treatment solution used in the other divisions of the same sterilization zone and/or the washing zone is supplied in series.

In each division of the sterilization or washing zone, a container is directly transferred from a sterilization or washing turret in a preceding stage to a turret in a next stage without being handed by a handing turret.

According to an aspect, piping is installed so that the sterilization zone and/or the washing zone to which the treatment solution is supplied first are/is on the most downstream side and that the treatment solution is successively supplied in series to the upstream zone.

### EFFECTS OF THE INVENTION

In the method for sterilizing and washing the container according to the invention, the sterilization step and/or the washing step determined by the sterilization or washing time (s) are/is divided into two or more (N divisions) and the treatment solution supplied to the divisions of the sterilization or washing step is stored after use and pumped up. Therefore, the amounts of the medicinal agent and the washing water to be supplied to each step may be reduced to 1/N and the tanks and the supply pumps to be disposed can be small. Therefore, even if the increase in the speed of the line is required, the device does not increase in size and equipment for general purpose use suffices, which reduces cost.

Because an amount of medicine to be supplied to each turret in the sterilization step maybe small, it is unnecessary to increase the size of a heat exchanger for maintaining the medicine at a predetermined temperature, which also reduces the cost. Because the treatment solution is supplied in series to the divisions of the same sterilization or washing step, it is possible to suppress an amount of the treatment solution to be used. Furthermore, aseptic water sterilized by heating is often used as the washing water and it is possible to similarly suppress the cost of the heating treatment.

In the method for sterilizing and washing the container according to the invention, because the sterilization step and the washing step are respectively divided into two or more (N) stages, intervals are provided between treatments in the respective stages and the treatment solution is temporarily discharged from each stage, which enhances sterilization performance and washing performance.

Even if the system is operated at a constant speed or even if a range of treatment to be carried out at one turret is determined in advance and a treatment time is fixed, it is possible to divide the treatment time by combining a plurality of rotaries.

### BRIEF DESCRIPTION OF THE DRAWINGS

FIG. 1 illustrates a structure of a system for sterilizing and washing a container according to the present invention.

FIG. 2 illustrates a conventional structure in which washing of a container is carried out in multiple stages.

FIG. 3 illustrates a structure of a conventional container washing system.

### EXPLANATION OF REFERENCE NUMMERALS

1 bottle carry-in room
2 washing room
3 aseptic room
4A, 4B bottle inverting device
5A, 5B, 5C sterilization washing turret
6A, 6B, 6C tank
7A, 7B, 7C pump
8A, 8B finish washing turret
9 handing turret
10 filling turret
11 capping turret

### MOOD FOR CARRYING OUT THE INVENTION

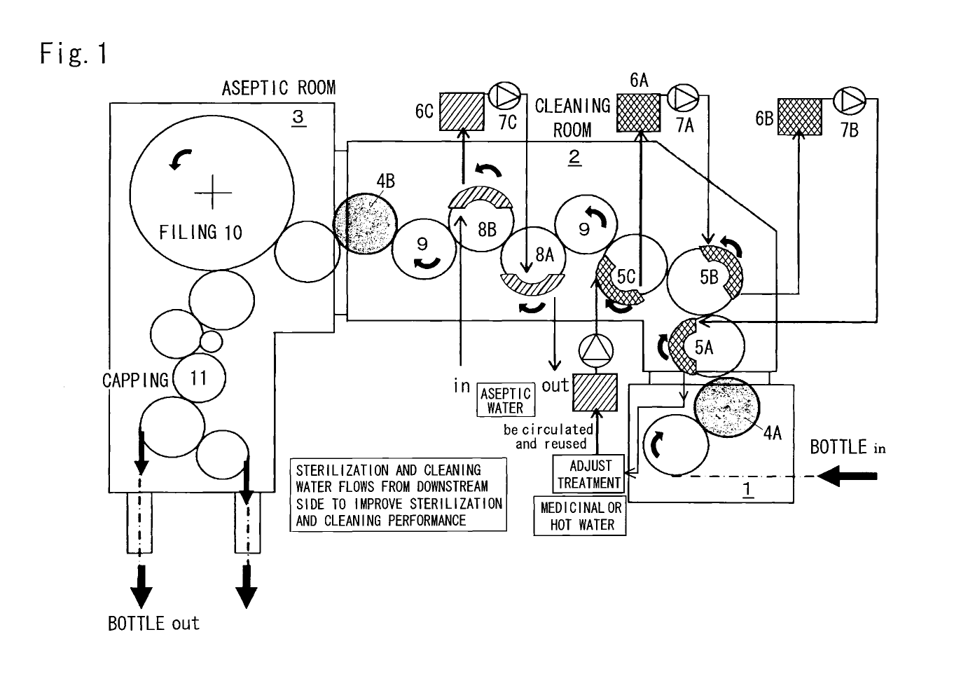

An embodiment of the present invention will be described below in detail. FIG. 1 illustrates a structure of a system for sterilizing and washing a container according to the invention. A container (bottle) sent in by a conveyor through an entrance of a bottle carry-in room 1 is retained upside down by a bottle inverting device 4A so that an opening portion is on a lower side and sent into a washing room 2 which is a sealed space on a downstream side. The washing room 2 is divided into a sterilization washing zone and a finish washing zone along a flow and sterilization washing turrets 5A, 5B, and 5C divided into a plurality of stages (three stages in this example) are directly connected in the sterilization washing zone. As sterilization treatment solution, a medicine such as peracetic acid solution, hydrogen peroxide solution, and sodium hypochlorite is used in a sterilization method, ozone water is used in a washing method, and only hot water is used to sterilize the bottle in a method without using the medicine as described above. In the sterilization zone, piping is installed so that the sterilization treatment solution is first supplied to the turret 5C on the most downstream side and that the treatment solution used at the turret 5C is recovered and stored in a tank 6A and transferred to the directly upstream turret 5B by a supply pump 7A. The sterilization washing is carried out here as well and the treatment solution after use is recovered and stored in a tank 6B and transferred to the directly upstream turret 5A by a supply pump 7B. The treatment solution used on the most upstream side is recovered. If the medicine is used as the sterilization treatment solution, concentration of the medicine is adjusted and the treatment solution is circulated and reused to be supplied to the first turret 5C on the most downstream side. The concentration of the medicine may be reduced during use in some cases and is adjusted to about 2500 ppm when the peracetic acid solution is used, for example.

This form in which the treatment solution is not supplied in parallel to the divisions of the sterilization washing zone but successively circulated in series is advantageous in that small tanks and pumps for general purpose use can be used though the respective turrets need to have individual tanks and pumps, because the treatment solution is separately supplied to the respective turrets. Because an amount of the treatment solution to be supplied in parallel as in a conventional case becomes large as the speed of the line increases, a pipe before a branch point needs to have a large diameter and requires a large pump. However, it is unnecessary to prepare the special pipe having the large diameter and the large pump in the present invention in which the treatment solution is supplied in series.

In the sterilization treatment, a nozzle is inserted into a mouth portion of the bottle, which opens downward as the turret turns, the treatment solution is sprayed upward, and the treatment solution is simultaneously sprayed on an outer face portion of the bottle from a plurality of nozzles to sterilize inner and outer faces of the bottle. When the spraying is finished, the used treatment solution flows downward and is drained out without remaining in the bottle and the bottle is sent to a step in the next stage and subjected to the sterilization treatments step by step in the respective stages, which increases a sterilization effect.

The bottle after the sterilization washing is conveyed to the finish washing zone via a handing turret 9. In the example shown in FIG. 1, the finish washing zone is divided into two stages and the bottle is finish-washed at the two turrets 8A and 8B. Aseptic water for washing is first supplied to the downstream turret 8B. This washing is a step for washing the bottle which has been washed and sterilized on the upstream side. Piping is installed so that the sterilization treatment solution used for washing at the downstream turret 8B is recovered and stored in a tank 6C and is transferred to the directly upstream turret 8A by a supply pump 7C. Here, the washing is carried out for the bottle to the inner and outer faces of which the sterilization treatment solution has adhered in the above-described sterilization washing zone. The aseptic water used for the washing twice is discharged and not circulated and reused. Along washing zone and a large amount of washing water are necessary to achieve finish washing for a time long enough to satisfy the manufacturing standards required by Food Sanitation Act when the speed of the line is increased. The above-described structure solves this problem by dividing the zone with the plurality of turrets and supplying the washing water to the respective stages in series.

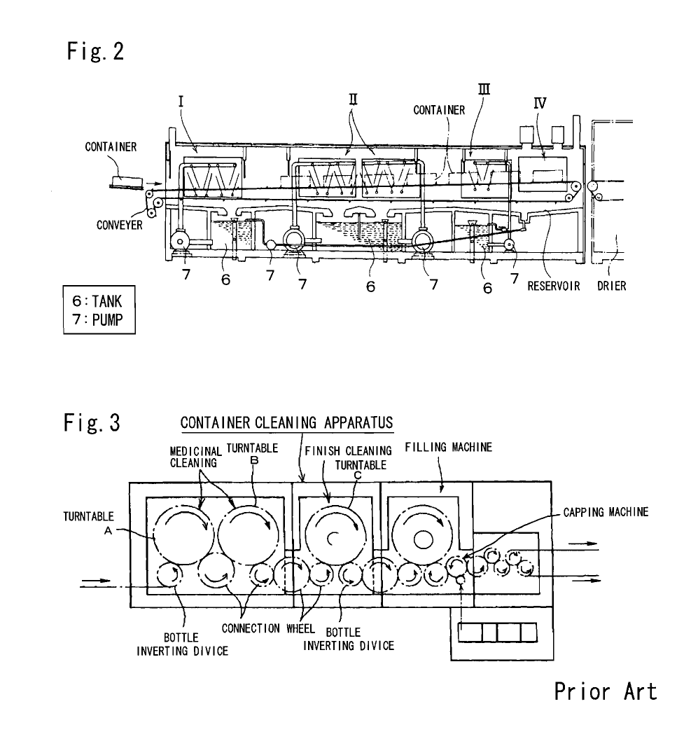

Patent Literature 2 discloses a method in which a washing step is formed by multiple stages in a general washing line and washing water is supplied from a downstream side and caused to flow successively upstream. This device consistently carries out steps from washing to predrying, increases washing efficiency in this series of steps, and achieves reuse of used washing water which conventionally used to be discharged uselessly in a rinse washing mechanism, suppression of heat energy consumption in a drying device, and efficient spraying of washing water. In this structure, as shown in FIG. 2, the respective washing mechanisms for pre-washing I, main washing II, and rinse washing III and draining mechanism IV by blowing of hot air are successively disposed along a conveying path. A plurality of washing water supply pipes having spray nozzles inside themselves and connected to each other are provided at least above and below the conveying path of each of the washing mechanisms, and the spray nozzles adjacent to each other in a conveying direction are inclined so that spraying directions of the spray nozzles are opposite to the conveying direction. A recovery mechanism for recovering waste water from the rinse washing mechanism and the draining mechanism is provided on a bottom side of the mechanisms, and a supply device for adding the water recovered by the recovery mechanism into the pre-washing mechanism is provided. Because the used washing water recovered from the relatively clean washing mechanism and draining mechanism is added into the heavily-polluted pre-cleaner vessel, it is more economical than supplying new washing water. Moreover, a temperature of the recovered washing water to be added is much higher than that of normal tap water and therefore less heating is necessary in the pre-washing mechanism, which is effective as energy-saving measures. However, in this device, the different treatments, i.e., the pre-washing, the main washing, and the clean washing respectively using different amounts of washing water are carried out successively and the used washing water, i.e., part of the washing water recovered from the clean washing mechanism and the draining mechanism is added into the heavily-polluted pre-cleaner vessel. Therefore, the device is different from the idea of the invention in which the finish washing step is divided and the treatment solution supplied to one division is stored after the use and pumped up to be supplied in series to the other division in the same washing step to thereby suppress increase in the treatment solution, and does not suggest such an idea.

In the sterilization and washing method of the invention, the finish washing zone is divided into the multiple stages and the bottle is successively finish-washed at the plurality of turrets from the upstream side. Because the bottle is washed with its mouth portion oriented downward and the finish washing is carried out independently in each stage, the washing water used here for the finish washing is cleanly discharged from each stage and it is possible to effectively carry out the washing in the next stage. The finish washing zone is divided into the two stages in the example shown in FIG. 1 and the aseptic water used for washing is first supplied to the downstream turret 8B. The piping is installed so that the washing water used here is recovered and stored in the tank 6C and is transferred to the directly upstream turret 8A by the supply pump 7C and the washing water is used for the washing at the turret 8A. Because the washing water used for the finish washing on the most upstream side includes small amounts of ingredients of the medicine adhering to the inner and outer walls of the bottle, the washing water is not reused but discharged. Because the finish washing zone is divided into the two stages in this example, the washing water used at the turret 8A is discharged.

The bottle after the finish washing is handed to a bottle inverting device 4B by the handing turret 9 and the inverted bottle is returned into the erected attitude and conveyed from the washing room 2 to a next aseptic room 3. In the aseptic room 3, content is filled, capping is carried out by using a cap separately sterilized and washed, and the bottle is discharged as a finished product of the beverage filled in the container.

In the invention, the sterilization washing step (sterilization step) and/or the finish washing step (washing step) are/is respectively divided into a plurality of stages to effectively utilize the space and the treatment solution is successively transferred in series to the respective stages to reduce the amount of the treatment solution to adapt to the increase in the speed of the line. The number of divisions, whether the sterilization is carried out by the medicinal agent treatment solution, whether the washing or the washing with the ozone water is carried out after the sterilization, and whether the washing is carried out by using the hot water for the sterilization as necessary in the sterilization and washing are determined suitably according to the kind of content to be filled, i.e., acidic beverage or tea.

In the case of the sterilization with the treatment solution including the medicine, it is sometimes necessary to maintain the temperature. Because the installation which can be realized by the above-described structure can be small, it is advantageously possible to use small heat-exchange equipment.

1. A method for sterilizing and washing a container, comprising:
dividing a sterilization step and/or a washing step determined by a sterilizing or washing time into two or more, and
storing treatment solution supplied to a division of the sterilization step and/or the washing step after use and pumping up the treatment solution to thereby supply the treatment solution in series to the other divisions of the same sterilization step and/or the washing step.

2. The method for sterilizing and washing the container according to claim 1, wherein the treatment solution supplied to a most downstream division of the sterilization step and/or the washing step is stored after use and pumped up, the treatment solution supplied to an adjacent upstream division of the same step is stored again after use and pumped up, and the treatment solution is successively supplied to the upstream divisions of the same step, so that the treatment solution is supplied in series to the all divisions of the step.

3. The method for sterilizing and washing the container according to claim 1, wherein the kind of the treatment solution and the number of divisions of the sterilization step and the number of divisions of the washing step are selected according to the kind of beverage liquid.

4. The method for sterilizing and washing the container according to claim 2, wherein the kind of the treatment solution and the number of divisions of the sterilization step and the number of divisions the washing step are selected according to the kind of beverage liquid.

5. A system for sterilization and washing a container, wherein a sterilization zone and/or a washing zone determined by a sterilizing or washing time are/is divided into two or more, a tank and a pump for supplying treatment solution to each division of the sterilization or washing zone are disposed, and, to the tanks for supplying the treatment solution to the respective zones excluding the one tank for supplying the treatment solution first, the treatment solution used in the other divisions of the same sterilization zone and/or the washing zone is supplied in series.

6. The system for sterilization and washing the container according to claim 5, wherein, in each division of the sterilization or washing zone, a container is directly transferred from a sterilizing or washing turret in a preceding stage to a turret in a next stage without being handed by a handing turret.

7. The system for sterilization and washing the container according to claim 5, wherein piping is installed so that the sterilization zone and/or the washing zone to which the treatment solution is supplied first are/is on the most downstream side and that the treatment solution is successively supplied in series to the upstream zone.

8. The system for sterilization and washing the container according to claim 6, wherein piping is installed so that the sterilization zone and/or the washing zone to which the treatment solution is supplied first are/is on the most downstream side and that the treatment solution is successively supplied in series to the upstream zone.
